(19) United States
(12) Patent Application Publication
(10) Pub. No.: US 2021/0259656 A1
(43) Pub. Date: Aug. 26, 2021

(54) LIFESTYLE ASSESSMENT SYSTEM AND PROGRAM THEREOF

(71) Applicant: HIL CO., LTD., Kumamoto-shi, Kumamoto (JP)

(72) Inventor: Kenji IURA, Kumamoto-shi, Kumamoto (JP)

(21) Appl. No.: 17/255,764

(22) PCT Filed: Apr. 26, 2019
(86) PCT No.: PCT/JP2019/017959

§ 371 Date: Dec. 23, 2020

(51) Int. Cl. A61B 8/08; A61B 8/14; G16H 50/30; G06N 3/08

(52) U.S. Cl. CPC A61B 8/08; A61B 8/14; G06N 3/08; G16H 50/30; A61B 8/5207

(57) ABSTRACT

To appropriately assess lifestyle relating to metabolic syndrome. An ultrasonic probe captures an image of an abdomen of a subject and outputs a tomographic image of the abdomen. A feature assessing unit includes a learning model and a measuring unit, and outputs assessment index data indicating respective features of at least a subcutaneous fat layer, a visceral fat layer and right and left rectus abdominis muscle among living body portions visualized in the tomographic image. A measure presenting unit selectively presents one of a plurality of measure patterns obtained by systematically classifying measures concerning lifestyle, in accordance with the assessment index data.

### TECHNICAL FIELD

The present invention relates to a lifestyle assessment system and a program thereof, and more particularly, to assessment of lifestyle relating to metabolic syndrome.

### BACKGROUND ART

In related art, a method for diagnosing metabolic syndrome has been known. For example, Patent Literature 1 discloses a visceral fat estimation method for estimating visceral fat on the basis of an abdominal circumference at an umbilici position and a thickness of subcutaneous fat of an abdomen. Patent Literature 2 discloses a metabolic syndrome vessel assessment system which measures a preperitoneal fat thickness (PFT), an intima media thickness (IMT), and flow-mediated dilation (FMD) in an image of a portion to be inspected acquired with an ultrasonic probe, and diagnoses and assesses a degree of risk of metabolic syndrome by considering all these measurement results together.

Further, Patent Literature 3 discloses an ultrasonic system which measures an index value representing an amount of visceral fat in medical examination of metabolic syndrome. In this diagnosis system, first, a length a between a body surface and abdominal aorta, and a length a1 between an outer edge of a region including visceral fat and the abdominal aorta are measured in an abdominal tomographic image acquired with the ultrasonic probe. An abdominal circumference is measured separately from the above. An abdominal total area is computed from the abdominal circumference and the length a assuming ellipse approximation of an abdominal cross-section. Then, an area of the region including visceral fat is computed as an area (partial area) of a shape similar to an ellipse from the total area, the length a and the length a1, and the index value representing the amount of visceral fat is computed from the partial area, and one or a plurality of personal parameter values concerning a subject.

Further, Patent Literature 4 discloses an ultrasonic measurement device which obtains tissue thickness information including a thickness of muscle and a thickness of fat of the subject on the basis of an ultrasonic image, and generates guide information for making an index value of an amount of tissue of the subject closer to a target value on the basis of this tissue thickness information.

### CITATION LIST

### Patent Literature

Patent Literature 1: JP 2007-111166 A

Patent Literature 2: JP 2008-188077 A

Patent Literature 3: JP 2014-33816 A

Patent Literature 4: JP 2015-142619 A

### SUMMARY OF INVENTION

### Technical Problem

In recent years, health promotion has been encouraged in terms of suppression of medical expenses, and the like, and it is desired to improve daily lifestyle to reduce visceral fat so as to prevent development of metabolic syndrome, and the like, in the future. However, there is only a method for making a diagnosis as to whether or not he/she develops metabolic syndrome now, and there is no mechanism for improving lifestyle or giving guidance to people including people who has not yet developed metabolic syndrome, to prevent development of metabolic syndrome. It goes without saying that even a slender person may develop metabolic syndrome in the future if he/she continues to eat too much or lack exercise. It is therefore of great significance to assess lifestyle in a stage in which he/she has not developed metabolic syndrome yet and encourage improvement of lifestyle as necessary. The present inventor has observed abdomens and has interviewed twenty thousand or more subjects in 13 years at various sites such as medical settings, gyms and event venues, and has achieved an effective objective assessment method as a result of continuously verifying overeating and lack of exercise of slender people.

The present invention has been made in view of such circumstances, and an object of the present invention is to appropriately assess lifestyle relating to metabolic syndrome.

### Solution to Problem

In order to solve the above problem, a first invention includes an ultrasonic probe and a feature assessing unit, and provides a lifestyle assessment system which assesses lifestyle relating to metabolic syndrome. The ultrasonic probe captures an image of an abdomen of a subject and outputs a tomographic image of the abdomen. The feature assessing unit outputs data indicating respective features of at least a subcutaneous fat layer, a visceral fat layer and right and left rectus abdominis muscle among living body portions visualized in the tomographic image as assessment index data of lifestyle.

Here, in the first invention, a measure presenting unit may be provided. The measure presenting unit selectively presents one of a plurality of measure patterns obtained by systematically classifying measures concerning lifestyle, on the basis of the assessment index data.

In the first invention, the feature assessing unit may include a first learning model and a measuring unit. The first learning model identifies each of the subcutaneous fat layer, the visceral fat layer and the right and left rectus abdominis muscle visualized in the tomographic image. The measuring unit measures each of the subcutaneous fat layer, the visceral fat layer and the right and left rectus abdominis muscle identified with the first learning model in accordance with a predetermined criterion, and outputs the assessment index data on the basis of a plurality of measurement values obtained through the measurement. In this case, it is preferable to provide a first learning processing unit. The first learning processing unit performs learning processing of the first learning model through supervised learning using training data which gives an instruction of respective positions of the subcutaneous fat layer, the visceral fat layer and the right and left rectus abdominis muscle visualized in the tomographic image.

In the first invention, the feature assessing unit may include a second learning model. The second learning model classifies an integrated feature of the subcutaneous fat layer, the visceral fat layer and the right and left rectus abdominis muscle visualized in the tomographic image into one of a plurality of classification patterns defined in advance. The second learning model is included. The feature assessing unit outputs the assessment index data on the basis of the classification pattern classified with the second learning model. In this case, it is preferable to provide a second learning processing unit. The second learning model performs learning processing of the second learning model through supervised learning using training data which gives an instruction of a classification pattern into which an integrated feature of the subcutaneous fat layer, the visceral fat layer and the right and left rectus abdominis muscle visualized in the tomographic image is classified.

In the first invention, it is preferable that the assessment index data include a feature of a shape of the right and left rectus abdominis muscle, and quantitative features of the subcutaneous fat layer and the visceral fat layer. Further, the assessment index data may include brightness of the rectus abdominis muscle in the tomographic image. Furthermore, it is preferable that the tomographic image be acquired with the ultrasonic probe in a state where a subject raises his/her upper body up.

A second invention causes a computer to execute the following steps, and provides a lifestyle assessment program for assessing lifestyle relating to metabolic syndrome. A first step analyses a tomographic image acquired by an image of an abdomen of a subject being captured with an ultrasonic probe. A second step outputs data indicating respective features of at least a subcutaneous fat layer, a visceral fat layer, and right and left rectus abdominis muscle among living body portions visualized in the tomographic image as assessment index data of lifestyle.

Here, in the second invention, a third step may be provided. The third step selectively presents one of a plurality of measure patterns obtained by systematically classifying measures concerning lifestyle, on the basis of the assessment index data.

In the second invention, the first step may include a step of inputting a tomographic image acquired with the ultrasonic probe to a first learning model for identifying the subcutaneous fat layer, the visceral fat layer and the right and left rectus abdominis muscle visualized in the tomographic image, and a step of measuring each of the subcutaneous fat layer, the visceral fat layer and the right and left rectus abdominis muscle identified with the first learning model. In this case, in the second step, the assessment index data is output on the basis of a plurality of measurement values obtained through the measurement. Further, in this case, a fourth step of performing learning processing of the first learning model through supervised learning using training data which gives an instruction of respective positions of the subcutaneous fat layer, the visceral fat layer and the right and left rectus abdominis muscle visualized in the tomographic image may be provided.

In the second invention, the first step may include a step of inputting a tomographic image acquired with the ultrasonic probe to a second learning model for classifying an integrated feature of the subcutaneous fat layer, the visceral fat layer and the right and left rectus abdominis muscle visualized in the tomographic image into one of a plurality of classification patterns defined in advance. In this case, in the second step, the assessment index data is output on the basis of the classification pattern classified with the second learning model. Further, in this case, a fourth step of performing learning processing of the second learning model through supervised learning using training data which gives an instruction of a classification pattern into which an integrated feature of the subcutaneous fat layer, the visceral fat layer and the right and left rectus abdominis muscle visualized in the tomographic image is classified may be provided.

In the second invention, it is preferable that the assessment index data include a feature of a shape of the right and left rectus abdominis muscle, and

quantitative features of the subcutaneous fat layer and the visceral fat layer. Further, the assessment index data may include brightness of the rectus abdominis muscle in the tomographic image. Furthermore, it is preferable that the tomographic image be acquired with the ultrasonic probe in a state where a subject raises his/her upper body up.

### Advantageous Effects of Invention

According to the present invention, data indicating respective features of a subcutaneous fat layer, a visceral fat layer and right and left rectus abdominis muscle among living body portions visualized in the tomographic image is output as assessment index data of lifestyle. It is possible to appropriately assess lifestyle relating to metabolic syndrome for people who have not developed metabolic syndrome yet as well as people who have already developed metabolic syndrome by focusing attention on also right and left rectus abdominis muscle as well as a fat layer such as a subcutaneous fat layer and a visceral fat layer and comprehensively assessing these features.

### BRIEF DESCRIPTION OF DRAWINGS

FIG. 1 is a block diagram of a lifestyle assessment system according to a first embodiment.

FIG. 2 is an explanatory diagram of abdominal diagnosis of a subject.

FIG. 3 is an explanatory diagram of a measurement example of rectus abdominis muscle.

FIG. 4 is an explanatory diagram of brightness of the rectus abdominis muscle in a tomographic image.

FIG. 5 is a classification map of features of the rectus abdominis muscle.

FIG. 6 is an explanatory diagram of patterns of combination of advices of measures.

FIG. 7 is an explanatory diagram of details of the advices of measures.

FIG. 8 is a block diagram of a lifestyle assessment system according to a second embodiment.

### DESCRIPTION OF EMBODIMENTS

### First Embodiment

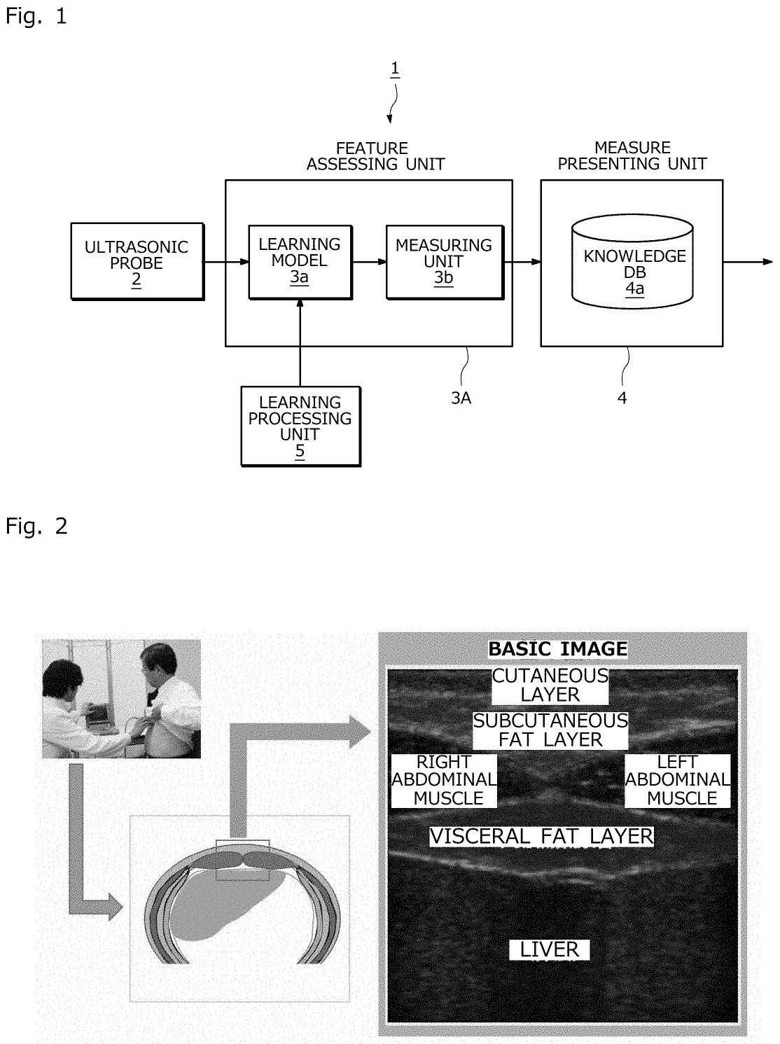

FIG. 1 is a block diagram of a lifestyle assessment system according to a first embodiment. This lifestyle assessment system 1 assesses features such as amounts and shapes of mainly four living body portions of a subcutaneous fat layer, a visceral fat layer, and right and left rectus abdominis muscle on the basis of a captured ultrasonic image (echo image) of inside of the abdomen of a subject, and outputs these features as assessment indexes of lifestyle. Here, among various kinds of assumed lifestyle, the present embodiment focuses attention on lifestyle relating to metabolic syndrome.

Further, the lifestyle assessment system according to the present embodiment also has a function of presenting an advice which is useful for improving lifestyle of the subject, and the like. Overeating, lack of exercise, and the like, are assessed in an objective manner so as to be able to be understood by everyone by structuring quantitative, qualitative or morphological change of subcutaneous fat, visceral fat and right and left rectus abdominis muscle. The present embodiment has a feature of focusing attention also on right and left rectus abdominis muscle as well as a subcutaneous fat layer and a visceral fat layer to assess lack of exercise, or the like, of the subject. Typically, the rectus abdominis muscle is not used in daily life compared to muscle of the hands and feet, and thus, it can be considered a person who has firm rectus abdominis muscle generally has firm muscle of four limbs. From this, it is possible to estimate a degree of lack of exercise of the subject by introducing features of the right and left rectus abdominis muscle as an assessment index.

The lifestyle assessment system 1 includes an ultrasonic probe 2, a feature assessing unit 3A, a measure presenting unit 4, and a learning processing unit 5. The ultrasonic probe 2 captures an image of the abdomen of the subject and acquires a tomographic image of the abdomen. FIG. 2 is an explanatory diagram of abdominal diagnosis of the subject. A person who makes a diagnosis captures a tomographic image of an abdomen by making the ultrasonic probe 1 abut on the abdomen of the subject. The example in FIG. 2 is a captured tomographic image of a portion near the liver, in which a subcutaneous fat layer positioned immediately below a cutaneous layer, a visceral fat layer positioned immediately above a peritoneum, and right and left rectus abdominis muscle (rectus abdominis muscle) positioned between the subcutaneous fat layer and the visceral fat layer are visualized.

Here, as illustrated in FIG. 2, it is preferable to capture a tomographic image in a state where the subject raises his/her upper body up. The present inventor has obtained knowledge as a result of error and trial over many years that change (variation) of living body portions is relatively less and a favorable tomographic image which is appropriate for diagnosis can be stably acquired in a state where the subject raises his/her upper body up than in a state where the subject lies down.

The tomographic image acquired with the ultrasonic probe 2 is output to the feature assessing unit 3A. The feature assessing unit 3A receives input of the tomographic image from the ultrasonic probe 2 and outputs assessment index data. This assessment index data indicates features of at least a subcutaneous fat layer, a visceral fat layer and right and left rectus abdominis muscle among living body portions visualized in the tomographic image and indicates indexes of these features.

In the present embodiment, the feature assessing unit 3A includes a learning model 3a and a measuring unit 3b. The learning model 3a is mainly constituted with a neural network and has predetermined ability to solve problems. Specifically, the learning model 3a identifies regions of the subcutaneous fat layer, the visceral fat layer and the right and left rectus abdominis muscle visualized in this tomographic image which is input (see FIG. 2). Here, the “neural network” is combination of mathematical models of neurons, and broadly includes the most primitive configuration as the neural network, and a derivative form and a developed form such as a convolutional neural network (CNN) and a recurrent neural network (RNN). Further, you only look once (YOLO), single shot multibox detector (SSD), or the like, which has recently attracted attention as neural network object detection algorithm, may be used.

The learning model 3a has a predetermined function (Y=f(X, θ)) and an internal parameter θ of the predetermined function, for example, a connection weight of the neural network is adjusted in advance through preliminary learning so that a living body portion (a subcutaneous fat layer, a visceral fat layer and right and left rectus abdominis muscle) on which attention should be focused can be appropriately identified in a tomographic image which is input.

The learning processing unit 5 performs learning processing of the learning model 3a through supervised learning using training data which gives an instruction of respective positions of the subcutaneous fat layer, the visceral fat layer and the right and left rectus abdominis muscle visualized in the tomographic image. The internal parameter θ of the learning model 3a is adjusted through this learning processing. Repetition of supervised learning using a large amount and various kinds of training data optimizes the learning model 3a so that appropriate output can be obtained with respect to various kinds of input.

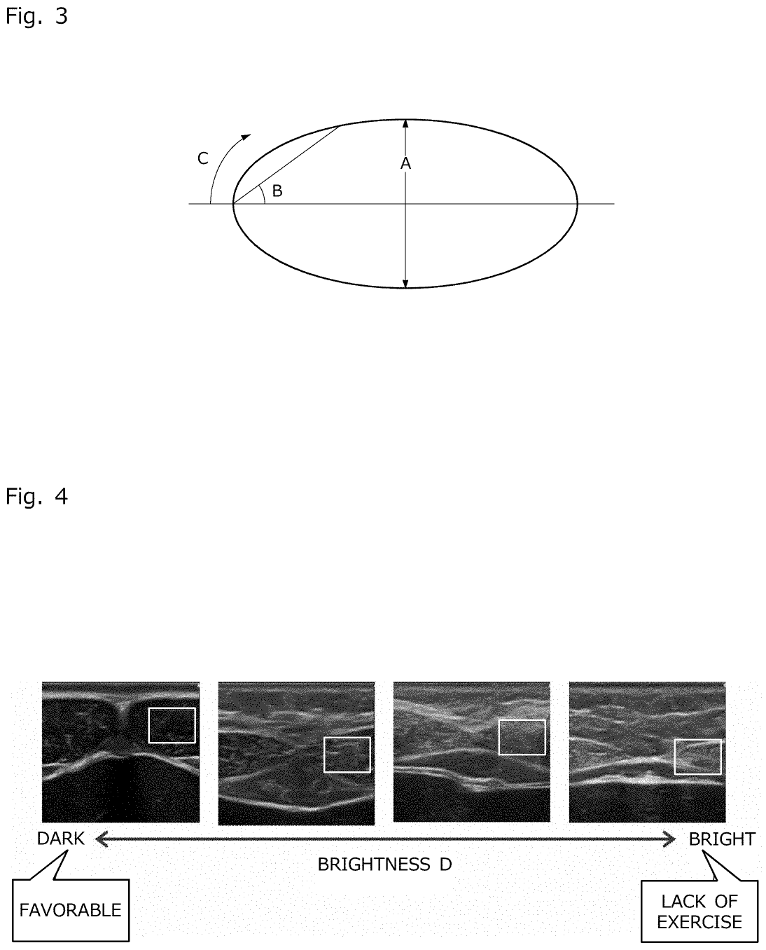

The measuring unit 3b measures features, specifically, features of a shape of the right and left rectus abdominis muscle whose regions are identified by the learning model 3a. FIG. 3 is an explanatory diagram of a measurement example of one of the rectus abdominis muscle. In the present embodiment, a thickness A, an angle B and a rise C are measured as the features of the shape of the rectus abdominis muscle. Here, the thickness A is a thickness of the rectus abdominis muscle, the angle B is an angle formed by a median line in a transverse section image of the rectus abdominis muscle and an apex of a bulge, and the rise C is a rising state from the median line in the transverse section image of the rectus abdominis muscle. Further, brightness D of the right and left rectus abdominis muscle visualized in the tomographic image may be measured as illustrated in FIG. 4 in addition to the measurement values A to C of the features of the shape. Typically, as muscle weakens or a period during which muscle is not actively used becomes longer, the brightness D of the rectus abdominis muscle becomes higher (becomes white). It is therefore possible to infer a health state of the muscle by setting a state of the brightness of the rectus abdominis muscle as the brightness D and assessing the brightness D. These measurement values A to D are calculated for each of the right and left rectus abdominis muscle.

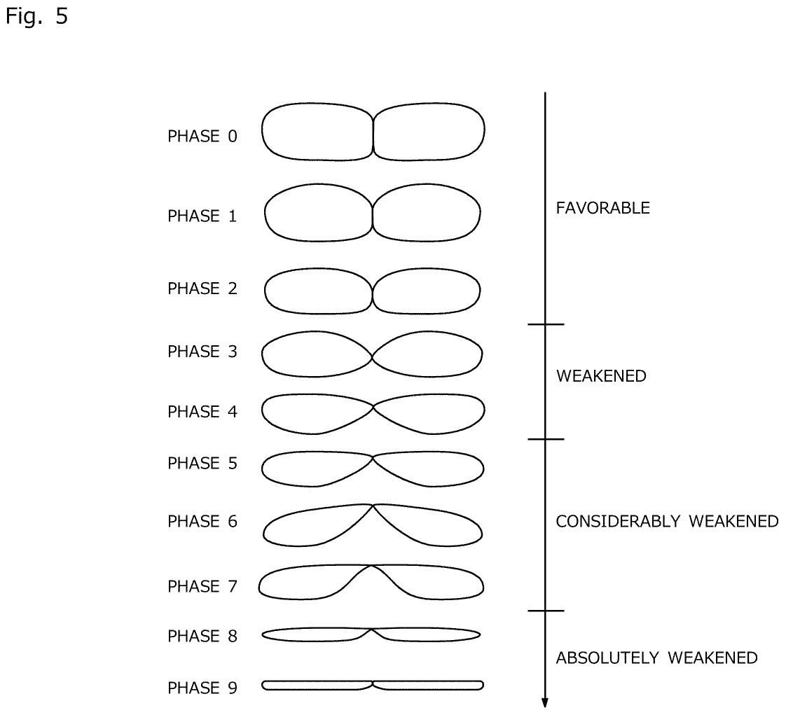

FIG. 5 is a classification map of features of the rectus abdominis muscle. The features (state) of the shape of the rectus abdominis muscle are classified into one of a plurality of phases defined in advance on the basis of the measurement values A to D of the rectus abdominis muscle. In FIG. 5, a phase 0 indicates a state where the rectus abdominis muscle is the most favorable (exercise is sufficient), and, as the phase becomes greater, the state of the rectus abdominis muscle gradually weakens (the subject is more likely to lack exercise), and a phase 9 indicates a state where the rectus abdominis muscle is the weakest (the subject completely lacks exercise). The phase representing the features of the shape of the rectus abdominis muscle is output to the measure presenting unit 4 as part of the assessment index data.

Further, the measuring unit 3b individually measures features of the subcutaneous fat layer and the visceral fat layer whose regions are identified by the learning model 3a, specifically, measures quantitative features (states) of these fat layers. The quantitative feature of the subcutaneous fat layer is classified into one of a plurality of phases defined in advance on the basis of a measurement value (for example, a thickness) of the subcutaneous fat layer. In a similar manner, the quantitative feature (state) of the visceral fat layer is classified into one of a plurality of phases defined in advance on the basis of a measurement value (for example, a thickness) of the visceral fat layer. Respective phases into which the features of the subcutaneous fat layer and the visceral fat layer are classified are output to the measure presenting unit 4 as part of the assessment index data.

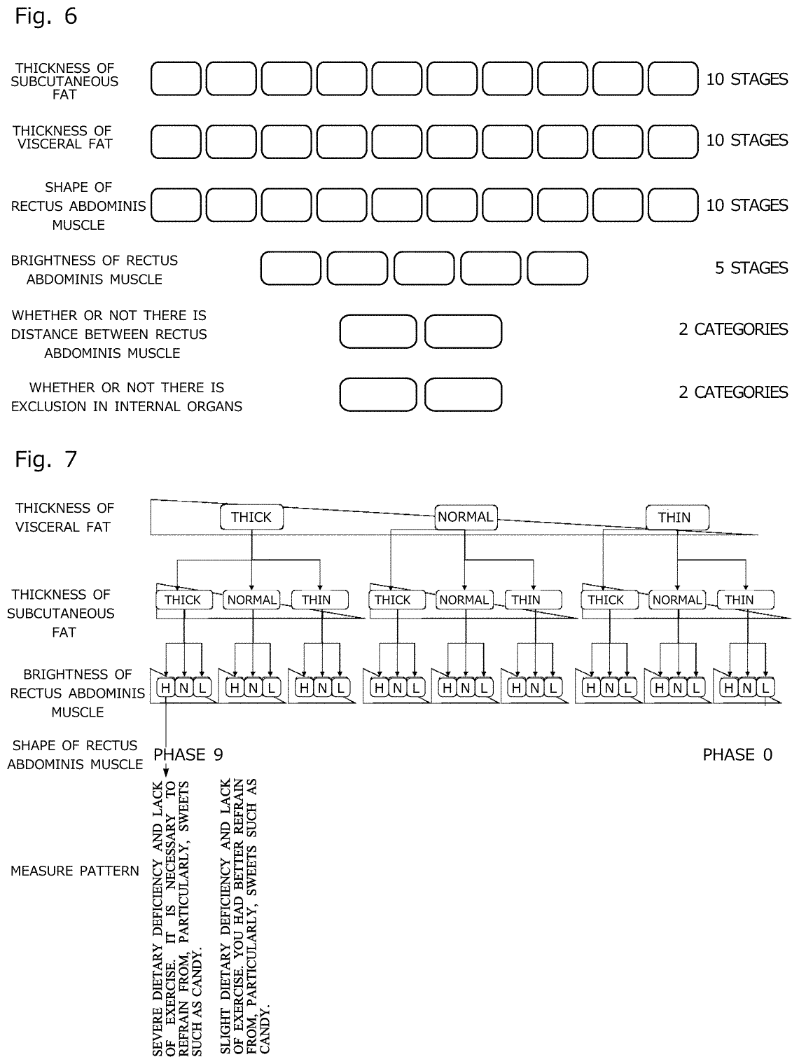

The measure presenting unit 4 presents a measure pattern concerning lifestyle to the subject in accordance with the assessment index data output from the feature assessing unit 3A. As illustrated in FIG. 6, the assessment index data only requires to include at least a “thickness of the subcutaneous fat” (10 stages) representing the quantitative feature of the visceral fat layer, a “thickness of the visceral fat” (10 stages) representing the quantitative feature of the visceral fat layer, a “shape of the rectus abdominis muscle” (10 stages) representing the feature of the shape of the rectus abdominis muscle (three-dimensional vector). In the present embodiment, in addition to these, the assessment index data includes “brightness of the rectus abdominis muscle” (5 stages) representing a state of brightness of the rectus abdominis muscle, “whether or not there is a distance between the rectus abdominis muscle” (2 categories), and “whether or not there is exclusion of internal organs” (2 categories) (six-dimensional vector). This makes it possible to present possible causes which can be inferred and measures as a measure pattern with respect to twenty thousand combinations. Note that while it is possible to set up to twenty thousand measure patterns, it is also possible to set less measure patterns by merging stages and categories on the vector in view of implementation.

The measure presenting unit 4 includes a knowledge database 4a. In this knowledge database 4a, a number of measure patterns which are obtained by systematically classifying measures regarding lifestyle are stored, and one of the measure patterns is selectively presented in accordance with the assessment index data. As illustrated in FIG. 7, details of advices of measures are individually defined in accordance with the “thickness of the visceral fat”, the “thickness of the subcutaneous fat”, the “brightness of the rectus abdominis muscle”, the “shape of the rectus abdominis muscle”, and the like. Here, concerning the “thickness of the visceral fat”, accumulation of the visceral fat is considered to be directly linked to lifestyle deceases and correlates with lack of exercise and ingestion of alcohol, fat and sweets. The “thickness of the subcutaneous fat” less fluctuates than the visceral fat, tends not to decrease by training of muscle, and tends to decrease by aerobic exercise, and the like, and a person who ingests sweets tends to have a thicker subcutaneous fat layer. Concerning the “brightness of the rectus abdominis muscle”, as the brightness is higher in echocardiography, it can be considered that the rectus abdominis muscle includes more fat, and the like, and as the brightness is lower, it can be considered that the rectus abdominis muscle includes only pure muscle. The “shape of the rectus abdominis muscle” is as described above.

Details of advices of measures to be presented to the subject are different depending on a pattern into which the features are classified in accordance with the assessment index data. This pattern includes, for example, an “ideal pattern”, an “athletic pattern”, a “male metabolic syndrome pattern”, a “female metabolic syndrome pattern”, an “asymmetrical pattern”, a “pattern of separated rectus abdominis muscle”, a “stodginess pattern”, a “short-term overeating pattern”, and the like.

Specifically, it can be said that the “ideal pattern” is an ideal state with less subcutaneous fat and less visceral fat, with thick rectus abdominis muscle and with a distinct narrow part. The “athletic pattern” is a pattern with less subcutaneous fat and less visceral fat, and with very thick rectus abdominis muscle which has a trapezoidal shape. The “male metabolic syndrome pattern” is a pattern with thick visceral fat, which indicates unfavorable dietary habits, and indicates a state where the weakened rectus abdominis muscle extends to right and left by increased visceral fat and makes a big belly. The “female metabolic syndrome pattern” is a state where the rectus abdominis muscle is thin and weakens, the right and left rectus abdominis muscle has no narrow part, extends straight and is connected, the brightness of the rectus abdominis muscle is high, and it is inferred that the subject severely lacks exercise. The “asymmetric pattern” is a pattern with thick subcutaneous fat and thick visceral fat, indicating unfavorable lifestyle, and with a difference in thickness between the right and left rectus abdominis muscle, indicating that there is a problem in how to use the body. The “pattern of separated rectus abdominis muscle” is a pattern for a person who is slender, but, in a case where a distance between the right and left rectus abdominis muscle is large, indicating that there is a possibility that lateral rectus abdominis muscle is strongly pulled outward from the rectus abdominis muscle and indicating a person whose waist line tends to be less curved. The “stodginess pattern” is a pattern for a person who does not have a big belly because of stiff muscle, but whose excessive visceral fat excludes internal organs, which makes a state where the stomach easily feels heavy. The “short-term overeating pattern” is a pattern for a person whose total amount of visceral fat is small, but eats too much in a very short term, which results in exclusion of internal organs by precipitously increased visceral fat and makes a state where the stomach easily feels heavy.

Thus, according to the present embodiment, data indicating respective features of a subcutaneous fat layer, a visceral fat layer and right and left rectus abdominis muscle among living body portions visualized in the tomographic image is output as assessment index data of lifestyle. Features of the fat layers such as the subcutaneous fat layer and the visceral fat layer become assessment indexes of overeating, stodginess, and the like, of the subject. Further, features of the right and left rectus abdominis muscle become assessment indexes of lack of exercise, and the like, of the subject. In the present embodiment, attention is also focused on the right and left rectus abdominis muscle in addition to the fat layers which are main portions on which attention has been focused in diagnosis of metabolic syndrome, and these features are comprehensively assessed. This makes it possible to appropriately assess lifestyle relating to metabolic syndrome for people who have not developed metabolic syndrome yet as well as people who have already developed metabolic syndrome.

Further, according to the present embodiment, one of a plurality of measure patterns obtained by systematically classifying measures concerning lifestyle, on the basis of the assessment index data of lifestyle is selectively presented. This makes it possible to automatically present objective and effective advices of measures concerning improvement, or the like, of lifestyle relating to metabolic syndrome.

Further, according to the present embodiment, use of the learning model 3a enables a subcutaneous fat layer, a visceral fat layer and right and left rectus abdominis muscle visualized in a tomographic image to be respectively identified with high accuracy, so that it is possible to appropriately present advices of measures with high reliability.

Still further, according to the present embodiment, by a tomographic image being acquired using the ultrasonic probe 2 in a state where the subject raises his/her upper body up, it is possible to reduce change (variation) of living body portions in the tomographic image, so that it is possible to stably acquire a favorable tomographic image which is appropriate for diagnosis.

### Second Embodiment

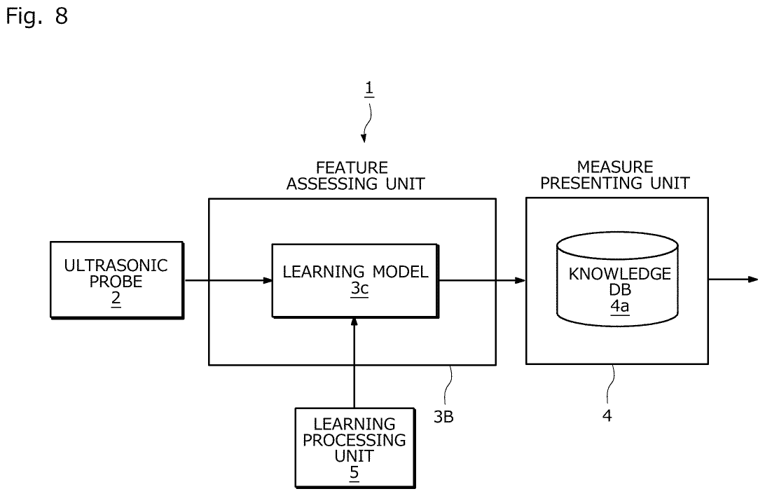

FIG. 8 is a block diagram of a lifestyle assessment system according to a second embodiment. The present embodiment has features in a configuration of a feature assessing unit 3B, specifically, in a configuration where the functions of the learning model 3a and the measuring unit 3b according to the first embodiment are integrally implemented with a single learning model 3c. Other points are similar to those in the first embodiment, and thus, the same reference numerals will be assigned, and description will be omitted here.

The learning model 3c classifies an integrated feature of the subcutaneous fat layer, the visceral fat layer and the right and left rectus abdominis muscle visualized in the tomographic image into one of a plurality of classification patterns defined in advance. As described above, this classification pattern includes at least the “thickness of the subcutaneous fat” (10 stages), the “thickness of the visceral fat” (10 stages) representing the quantitative feature of the visceral fat layer, a “shape of the rectus abdominis muscle” (10 stages) representing the feature of the shape of the rectus abdominis muscle (three-dimensional vector). Further, in addition to these, the classification pattern may include “brightness of the rectus abdominis muscle” (5 stages) representing a state of brightness of the rectus abdominis muscle, “whether or not there is a distance between the rectus abdominis muscle” (2 categories), and “whether or not there is exclusion of internal organs” (2 categories) (six-dimensional vector). Data classified with such a multidimensional vector is output to the measure presenting unit 4 as assessment index data. The measure presenting unit 4 presents a measure pattern on the basis of the assessment index data using a method similar to that in the first embodiment.

The learning processing unit 5 performs learning processing for the learning model 3c. However, content of output is different between the learning models 3a and 3c, and thus, training data for supervised learning is different. Specifically, training data which gives an instruction of a classification pattern into which an integrated feature of the subcutaneous fat layer, the visceral fat layer and the right and left rectus abdominis muscle visualized in the tomographic image is classified is used as data for a learning model 5c.

In this manner, according to the present embodiment, it is possible to provide operational effects similar to those in the above-described first embodiment and reduce processing load by integrating the learning model 3a and the measuring unit 3c according to the first embodiment into a single learning model 3c.

Note that, in the above-described respective embodiments, the measure presenting unit 4 does not necessarily have to be provided. For example, in a case where an adviser gives advice of measures regarding lifestyle to the subject with reference to the above-described assessment index data, it is not necessary to provide the measure presenting unit 4. Further, in a case where the lifestyle assessment system 1 is made to coordinate with an external system, it is not necessary to provide the measure presenting unit 4. For example, the lifestyle assessment system 1 is made to coordinate with a diagnosis system for arteriosclerosis or a circulatory system, and the assessment index data of the lifestyle assessment system 1 is used as one element for this diagnosis.

Further, the present invention can be regarded as functional blocks which constitute the lifestyle assessment system according to the above-described respective embodiments, specifically, a computer program (program for presenting measures for lifestyle) which equivalently implements the feature assessing units 3A and 3B and the measure presenting unit 4 with a computer.

### REFERENCE SIGNS LIST

1 Lifestyle assessment system
2 Ultrasonic probe
3A, 3B Feature assessing unit
3a, 3c Learning model
3b Measuring unit
4 Measure presenting unit
4a Knowledge database
5 Learning processing unit

1-18. (canceled)

19. A lifestyle assessment system which assesses lifestyle relating to metabolic syndrome, the lifestyle assessment system comprising:
a feature assessing unit configured to identify each of regions of right and left rectus abdominis muscle and a region of a predetermined fat layer among living body portions visualized in a tomographic image acquired by an image of an abdomen of a subject being captured with an ultrasonic probe, and output at least rectus abdominis muscle assessment index data indicating a feature of a shape of the right and left rectus abdominis muscle and fat layer assessment index data indicating a quantitative feature of the fat layer as assessment index data of lifestyle; and
a measure presenting unit including a knowledge database in which the feature of the shape of the right and left rectus abdominis muscle, the quantitative feature of the fat layer and measure patterns obtained by systematically classifying measures concerning lifestyle are associated and configured to selectively present one of the measure patterns by inputting the rectus abdominis muscle assessment index data and the fat layer assessment index data to the knowledge database.

20. The lifestyle assessment system according to claim 19,
wherein the fat layer assessment index data includes first fat layer assessment index data indicating a quantitative feature of a subcutaneous fat layer, and second fat layer assessment index data indicating a quantitative feature of a visceral fat layer,
in the knowledge database, the quantitative feature of the subcutaneous fat layer, the quantitative feature of the visceral fat layer, and the measure patterns are associated, and
the measure presenting unit inputs the first fat layer assessment index data and the second fat layer assessment index data to the knowledge database as the fat layer assessment index data.

21. The lifestyle assessment system according to claim 19, wherein the rectus abdominis muscle assessment index data includes a thickness of rectus abdominis muscle, an angle formed by a median line in a transverse section image of the rectus abdominis muscle and an apex of a bulge, and a rise indicating a rising state from the median line in the transverse section image of the rectus abdominis muscle.

22. The lifestyle assessment system according to claim 19, wherein the feature assessing unit includes:
a first learning model for identifying each of regions of the right and left rectus abdominis muscle and a region of the fat layer visualized in the tomographic image; and
a measuring unit configured to measure each of the regions of the right and left rectus abdominis muscle and the region of the fat layer identified with the first learning model, in accordance with a predetermined criterion, and output the assessment index data on a basis of a plurality of measurement results obtained through the measurement.

23. The lifestyle assessment system according to claim 22, further comprising: a first learning processing unit configured to perform learning processing of the first learning model through supervised learning using training data which gives an instruction of respective positions of the regions of the right and left rectus abdominis muscle and the region of the fat layer visualized in the tomographic image.

24. The lifestyle assessment system according to claim 19,
wherein the feature assessing unit includes:
a second learning model for classifying an integrated feature regarding a shape of the right and left rectus abdominis muscle and an amount of the fat layer visualized in the tomographic image into one of a plurality of classification patterns defined in advance, and
the feature assessing unit outputs the assessment index data on a basis of the classification pattern into which the integrated feature is classified with the second learning model.

25. The lifestyle assessment system according to claim 24, further comprising: a second learning processing unit configured to perform learning processing of the second learning model through supervised learning using training data which gives an instruction of the classification pattern into which the integrated feature regarding the shape of the right and left rectus abdominis muscle and the amount of the fat layer visualized in the tomographic image is classified.

26. The lifestyle assessment system according to claim 19,
wherein the assessment index data includes brightness assessment index data indicating brightness of the rectus abdominis muscle in the tomographic image,
in the knowledge database, the brightness of the rectus abdominis muscle and the measure patterns are associated, and
the measure presenting unit inputs the brightness assessment index data to the knowledge database.

27. The lifestyle assessment system according to claim 19, wherein the tomographic image is acquired with the ultrasonic probe in a state where a subject raises his/her upper body up.

28. A lifestyle assessment program for assessing lifestyle relating to metabolic syndrome, the lifestyle assessment program causing a computer to execute processing comprising:
a first step of identifying each of regions of right and left rectus abdominis muscle and a region of a predetermined fat layer among living body portions visualized in a tomographic image acquired by an image of an abdomen of a subject being captured with an ultrasonic probe, and outputting at least rectus abdominis muscle assessment index data indicating a feature of a shape of the right and left rectus abdominis muscle and fat layer assessment index data indicating a quantitative feature of the fat layer as assessment index data of lifestyle; and
a second step of selectively presenting one of measure patterns by inputting the rectus abdominis muscle assessment index data and the fat layer assessment index data to a knowledge database in which the feature of the shape of the right and left rectus abdominis muscle, the quantitative feature of the fat layer, and the measure patterns obtained by systematically classifying measures concerning lifestyle are associated.

29. The lifestyle assessment system according to claim 28,
wherein the fat layer assessment index data includes first fat layer assessment index data indicating a quantitative feature of a subcutaneous fat layer and second fat layer assessment index data indicating a quantitative feature of a visceral fat layer,
in the knowledge database, the quantitative feature of the subcutaneous fat layer, the quantitative feature of the visceral fat layer, and the measure patterns are associated, and
in the second step, the first fat layer assessment index data and the second fat layer assessment index data are input to the knowledge database as the fat layer assessment index data.

30. The lifestyle assessment program according to claim 28, wherein the rectus abdominis muscle assessment index data includes a thickness of rectus abdominis muscle, an angle formed by a median line in a transverse section image of the rectus abdominis muscle and an apex of a bulge, and a rise indicating a rising state from the median line in the transverse section image of the rectus abdominis muscle.

31. The lifestyle assessment program according to claim 28,
wherein the first step includes:
a step of inputting the tomographic image acquired with the ultrasonic probe to a first learning model for identifying each of regions of the right and left rectus abdominis muscle and a region of the fat layer visualized in the tomographic image; and
a step of measuring each of the regions of the right and left rectus abdominis muscle and the region of the fat layer identified with the first learning model, in accordance with a predetermined criterion, and
in the second step, the assessment index data is output on a basis of a plurality of measurement results obtained through the measurement.

32. The lifestyle assessment program according to claim 31, further comprising: a third step of performing learning processing of the first learning model through supervised learning using training data which gives an instruction of respective positions of the regions of the right and left rectus abdominis muscle and the region of the fat layer visualized in the tomographic image.

33. The lifestyle assessment program according to claim 28,
wherein the first step includes:
a step of inputting the tomographic image acquired with the ultrasonic probe to a second learning model for classifying an integrated feature regarding a shape of the right and left rectus abdominis muscle and an amount of the fat layer visualized in the tomographic image into one of a plurality of classification patterns defined in advance, and
in the second step, the assessment index data is output on a basis of the classification pattern classified with the second learning model.

34. The lifestyle assessment program according to claim 33, further comprising: a fourth step of performing learning processing of the second learning model through supervised learning using training data which gives an instruction of the classification pattern into which the integrated feature regarding the shape of the right and left rectus abdominis muscle and the amount of the fat layer visualized in the tomographic image is classified.

35. The lifestyle assessment program according to claim 28,
wherein the assessment index data includes brightness assessment index data indicating brightness of the rectus abdominis muscle in the tomographic image,
in the knowledge database, the brightness of the rectus abdominis muscle and the measure patterns are associated, and
in the second step, the brightness assessment index data is input to the knowledge database.

36. The lifestyle assessment program according to claim 28, wherein the tomographic image is acquired with the ultrasonic probe in a state where a subject raises his/her upper body up.
